(19) United States
(12) Patent Application Publication
(10) Pub. No.: US 2011/0275600 A1
(43) Pub. Date: Nov. 10, 2011

(54) METHODS FOR INCREASING ENDOGENOUS PLASMALOGEN LEVELS

(76) Inventors: Frederic Destaillats, Lutry (CH); Jean-Baptiste Bezelgues, Lucens (CH); Fabiola Dionisi, Epalinges (CH); Cristina Cruz-Hernandez, Epalinges (CH)

(21) Appl. No.: 13/138,066

(22) PCT Filed: Dec. 30, 2009
(86) PCT No.: PCT/US09/06749

§ 371 Date: Jun. 29, 2011

Related U.S. Application Data
(60) Provisional application No. 61/204,170, filed on Jan. 2, 2009.

(51) Int. Cl. A61K 31/661; A61K 31/232; A61P 35/00; A61P 25/28; A61P 25/00; A61K 31/202; A61P 3/00

(52) U.S. Cl. 514/120; 514/560; 514/549

(57) ABSTRACT

The invention provides methods for increasing endogenous plasmalogen levels in an animal by administering to the animal an endogenous plasmalogen level increasing amount of one or more long chain polyunsaturated fatty acids (LCPUFAs).

### CROSS REFERENCE TO RELATED APPLICATIONS

This application is a national stage application under 35 U.S.C. §371 of PCT/US2009/006749 filed Dec. 30, 2009, which claims priority to U.S. Provisional Application Ser. No. 61/204,170 filed Jan. 2, 2009, the disclosures of which are incorporated herein by reference.

### BACKGROUND OF THE INVENTION

1. Field of the Invention

The invention relates generally to methods for increasing endogenous plasmalogen levels in animals and particularly to methods for increasing endogenous plasmalogen levels in animals by administering long chain polyunsaturated fatty acids (LCPUFAs) to the animals.

2. Description of Related Art

Plasmalogens and their structure and use are known to skilled artisans. Plasmalogens are glycerol ether phospholipids wherein a glycerol moiety is bound to a 1-alkenyl ether group or a 1-alkyl ether group. Three major classes of plasmalogens have been identified and designated choline, ethanolamine, and serine plasmalogens. The chemical structure on one group of plasmalogens is shown in FIG. 1, which illustrates the ether linkage at C1 on the glycerol backbone. Typically, R is a hydrocarbon chain of varying length and X is choline, ethanolamine, or serine.

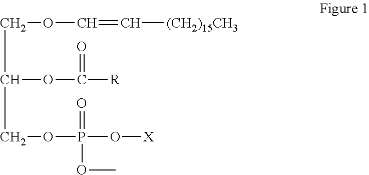

Plasmalogens are known to be associated with various diseases and conditions in animals, particularly in animals with low endogenous plasmalogen levels. Similarly, endogenous plasmalogen levels are known to decrease as an animal ages, possibly resulting in the onset of diseases and conditions adverse to the animal's health. U.S. Pat. No. 5,759,585 discloses the use of plasmalogens for treating neurodegenerative diseases. U.S. Pat. No. 6,177,476 discloses methods for replenishing plasmalogens in mammals using monoethers of glycerols and their carboxylic acid ester derivatives. WO08124916A1 discloses methods for the diagnosis and risk assessment of plasmalogen deficiency mediated diseases of aging, particularly colon cancer, prostate cancer, lung cancer, breast cancer, ovarian cancer, kidney cancer, cognitive impairment, and dementia.

Given the adverse effect of low endogenous plasmalogen levels on animals and their health, there is a need for novel methods for increasing endogenous plasmalogen levels in animals.

### SUMMARY OF THE INVENTION

It is, therefore, an object of the present invention to provide methods for increasing endogenous plasmalogen levels in animals.

It is another object of the present invention to provide methods for preventing or treating diseases or conditions in an animal caused or affected by decreased plasmalogen levels.

It is another object of the present invention to provide methods for extending the healthy prime years of an animal's life.

It is another object of the present invention to provide methods for promoting the health or wellness of an animal.

One or more of these other objects are achieved by administering an endogenous plasmalogen level increasing amount of one or more LCPUFAs to the animal. In various embodiments, LCPUFAs are administered to the animal in amounts of from about 1 to about 1000 milligrams per kilogram of body weight per day (mg/kg/day), preferably from about 5 to about 500 mg/kg/day, most preferably from about 10 to about 300 mg/kg/day. The methods are useful for preventing or treating diseases or conditions such as metabolic syndrome, neurodegenerative disease, dementia, Alzheimer's disease, cognitive impairment, colon cancer, prostate cancer, lung cancer, breast cancer, ovarian cancer, and kidney cancer, particularly in aging animals and more particularly in aging animals that are experiencing a decrease in endogenous plasmalogen levels.

Other and further objects, features, and advantages of the present invention will be readily apparent to those skilled in the art.

### DETAILED DESCRIPTION OF THE INVENTION

### Definitions

The term “animal” means any animal that could benefit from an increase in endogenous plasmalogen levels, including avian, bovine, canine, equine, feline, hicrine, murine, ovine, and porcine animals.

The term “long chain polyunsaturated fatty acid” or (“LCPUFA”) means any LCPUFA or compound that provides a LCPUFA when metabolized by an animal.

The term “plasmalogen agent” means any compound, composition, or drug other than LCPUFAs useful for increasing or decreasing endogenous plasmalogen levels in animals.

The term “in conjunction” means that one or more LCPUFAs, plasmalogen agents, or other compounds or compositions are administered to an animal (1) together in a food composition or (2) separately at the same or different frequency using the same or different administration routes at about the same time or periodically. “Periodically” means that the plasmalogen agent is administered on a dosage schedule acceptable for a specific plasmalogen agent and that the LCPUFAs are administered to an animal routinely as appropriate for the particular animal. “About the same time” generally means that the LCPUFAs and plasmalogen agent are administered at the same time or within about 72 hours of each other. “In conjunction” specifically includes administration schemes wherein plasmalogen agent is administered for a prescribed period and the LCPUFAs are administered indefinitely, particularly as part of a complete and balanced food composition.

The term “extending the prime” means extending the number of years an animal lives a healthy life and not just extending the number of years an animal lives, e.g., an animal would be healthy in the prime of its life for a relatively longer time.

The term “health and/or wellness of an animal” means the complete physical, mental, and social well being of the animal, not merely the absence of disease or infirmity.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein.

The term “single package” means that the components of a kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may be containers of individual plasmalogen agents and LCPUFAs or compositions containing LCPUFAs physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “an animal” or “a method” includes a plurality of such “animals” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the present invention provides methods for increasing endogenous plasmalogen levels in an animal. The methods comprise administering to the animal an endogenous plasmalogen level increasing amount of one or more long chain polyunsaturated fatty acids (LCPUFAs).

In another aspect, the invention provides methods for preventing or treating a disease or condition in an animal caused or affected by decreased plasmalogen levels. The methods comprise administering an endogenous plasmalogen level increasing amount of one or more LCPUFAs to an animal susceptible to or suffering from the disease or condition. The methods are useful for preventing or treating any disease caused or affected by decreased plasmalogen levels, as known to skilled artisans. In various embodiments, the methods are useful for preventing metabolic syndrome, neurodegenerative disease, dementia, Alzheimer's disease, cognitive impairment, colon cancer, prostate cancer, lung cancer, breast cancer, ovarian cancer, and kidney cancer.

Endogenous plasmalogen levels are decreased when they are below the average level known for an animal or species of animals. Average levels are known to skilled artisans.

In a further aspect, the present invention provides methods for extending the prime years of an animal's life. The methods comprise administering to the animal an endogenous plasmalogen level increasing amount of one or more LCPUFAs.

In another aspect, the invention provides methods for promoting the health or wellness of an animal. The methods comprise administering to the animal an endogenous plasmalogen level increasing amount of one or more LCPUFAs. The methods are useful for promoting health or wellness of animals of any age or classification, including adult animals and senior animals.

The LCPUFAs can be any LCPUFA known to skilled artisans. In various embodiments, the LCPUFAs are one or more monocarboxylic acids having at least 18 carbon atoms and at least two double bonds. In other embodiments, the LCPUFAs are one or more monocarboxylic acids having at least 20 carbon atoms and at least two double bonds. Examples of LCPUFAs include omega fatty acids such as linoleic acid 18:2 (n-6) all-cis-9,12-octadecadienoic acid; gamma-linolenic acid (GLA) 18:3 (n-6) all-cis-6,9,12-octadecatrienoic acid; eicosadienoic acid 20:2 (n-6) all-cis-11,14-eicosadienoic acid; dihomo-gamma-linolenic acid (DGLA) 20:3 (n-6) all-cis-8,11,14-eicosatrienoic acid; arachidonic acid (AA) 20:4 (n-6) all-cis-5,8,11,14-eicosatetraenoic acid; docosadienoic acid 22:2 (n-6) all-cis-13,16-docosadienoic acid; adrenic acid 22:4 (n-6) all-cis-7,10,13,16-docosatetraenoic acid; and docosapentaenoic acid (osbond acid) 22:5 (n-6) all-cis-4,7,10,13,16-docosapentaenoic acid and omega-3 fatty acids such as α-Linolenic acid (ALA) 18:3 (n-3) all-cis-9,12,15-octadecatrienoic acid; stearidonic acid (STD) 18:4 (n-3) all-cis-6,9,12,15-octadecatetraenoic acid; eicosatrienoic acid (ETE) 20:3 (n-3) all-cis-11,14,17-eicosatrienoic acid; eicosatetraenoic acid (ETA) 20:4 (n-3) all-cis-8,11,14,17-eicosatetraenoic acid; eicosapentaenoic acid (EPA) 20:5 (n-3) all-cis-5,8,11,14,17-eicosapentaenoic acid; docosapentaenoic acid (DPA) (clupanodonic acid) 22:5 (n-3) all-cis-7,10,13,16,19-docosapentaenoic acid; docosahexaenoic acid (DHA) 22:6 (n-3) all-cis-4,7,10,13,16,19-docosahexaenoic acid; tetracosapentaenoic acid 24:5 (n-3) all-cis-9,12,15,18,21-docosahexaenoic acid; and tetracosahexaenoic acid (nisinic acid) 24:6 (n-3) all-cis-6,9,12,15,18,21-tetracosenoic acid. LCPUFAs can be provided as free compounds or as esters such as acylglycerols (monoacylglycerol, diacylglycerol and triacylglycerol) or glycerophospholipids (phosphatidylcholine, phosphatidylethanolamine, phosphatidylinositol or phosphatidylserine). Preferably, the LCPUFAs are administered to the animal as triacylglycerols or phospholipids derivatives.

The LCPUFAs can be administered using any suitable method. Preferably, the LCPUFAs are administered to an animal orally. The LCPUFAs are administered orally using any suitable form for oral administration, e.g., tablets, pills, suspensions, solutions (possibly admixed with drinking water), emulsions, capsules, powders, syrups, and food compositions (a confectionery for a human, a pet food composition, or a treat or flavored treat for an animal). Preferably, the LCPUFAs are administered to the animal as part of a food composition. For animals such as companion animals, the LCPUFAs are administered as part of a complete and balanced diet.

The LCPUFAs can be administered to an animal in any amount useful for increasing endogenous plasmalogen levels. In various embodiments, LCPUFAs are administered to the animal in amounts of from about 1 to about 1000 milligrams per kilogram of body weight per day (mg/kg/day), preferably from about 5 to about 500 mg/kg/day, most preferably from about 10 to about 300 mg/kg/day. LCPUFAs can be administered in the diet, preferably a complete and balanced diet, in amounts of from about 1 to about 1000 grams per kilogram of diet (g/kg/diet), preferably from about 5 to about 500 g/kg/diet, most preferably from about 10 to about 300 g/kg/diet.

Generally, plasmalogen levels are increased in various tissues and organs, as known to skilled artisans. In one embodiment, plasmalogen levels are increased in brain glial cells of the animal, particularly adult and aging animals. In others, plasmalogen levels are increased in plasma lipids, lipoproteins, kidney, lung, testes, skeletal muscle, heart, brain, lymphocytes, spleen, macrophages, polymorphonuclear leukocytes, retina, and erythrocytes.

In a further aspect, the invention provides kits suitable for increasing endogenous plasmalogen levels in an animal. The kits comprise in separate containers in a single package or in separate containers in a virtual package, as appropriate for the kit component, one or more LCPUFAs and at least one of (1) one or more comestible ingredients suitable for consumption by an animal; (2) one or more plasmalogen agents; (3) instructions for how to combine two or more of the LCPUFAs, plasmalogen agents, and comestible ingredients to produce a composition suitable for consumption by an animal, particularly to produce a composition useful for increasing endogenous plasmalogen levels, preventing or treating diseases or conditions in an animal caused or affected by decreased plasmalogen levels, extending the healthy prime years of an animal's life, or promoting the health or wellness of an animal; (4) instructions for how to administer LCPUFAs and plasmalogen agents in conjunction, particularly to produce a composition useful for increasing endogenous plasmalogen levels, preventing or treating diseases or conditions in an animal caused or affected by decreased plasmalogen levels, extending the healthy prime years of an animal's life, or promoting the health or wellness of an animal; (5) one or more devices for mixing kit components; and (6) one or more devices for containing kit components.

When the kit comprises a virtual package, the kit is limited to instructions in a virtual environment in combination with one or more physical kit components. The kit contains LCPUFAs and other components such as plasmalogen agents in amounts sufficient for increasing endogenous plasmalogen levels in an animal. The kits may contain the kit components in any of various combinations and/or mixtures. In one embodiment, the kit contains a packet containing one or more LCPUFAs and a container of food for consumption by an animal, e.g., a dog or cat food or an animal treat. In another, the kit contains a packet containing one or more LCPUFAs and a plasmalogen agent. In a further, the kit contains a packet containing one or more LCPUFAs, a plasmalogen agent, and a container of food for consumption by an animal. Typically, the foods, plasmalogen agents, and LCPUFAS are admixed just prior to consumption. The kit may contain additional items such as a device for mixing LCPUFAs, plasmalogen agents, or comestible ingredients or a device for containing the admixture, e.g., a spoon and a food bowl.

In another aspect, the invention provides a means for communicating information about or instructions for one or more of (1) using LCPUFAs for increasing endogenous plasmalogen levels in an animal; (2) using LCPUFAs for preventing or treating a disease or condition in an animal caused or affected by decreased plasmalogen levels, e.g., metabolic syndrome, neurodegenerative disease, dementia, Alzheimer's disease, cognitive impairment, colon cancer, prostate cancer, lung cancer, breast cancer, ovarian cancer, and kidney cancer; (3) using LCPUFAs for extending the prime years of an animal's life; (4) using LCPUFAs for promoting the health or wellness of an animal; (5) admixing LCPUFAs with one or more comestible ingredients, particularly to produce a composition useful for increasing endogenous plasmalogen levels, preventing or treating diseases or conditions in an animal caused or affected by decreased plasmalogen levels, extending the healthy prime years of an animal's life, or promoting the health or wellness of an animal; and (6) using LCPUFAs in conjunction with one or more plasmalogen agents for increasing endogenous plasmalogen levels, preventing or treating diseases or conditions in an animal caused or affected by decreased plasmalogen levels, extending the healthy prime years of an animal's life, or promoting the health or wellness of an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

Useful information includes one or more of (1) methods and techniques for combining and administering LCPUFAs, plasmalogen agents, comestible ingredients, and other kit components and (2) contact information for animals or their caregivers to use if they have a question about the invention and its use. Useful instructions include amounts for mixing and administration amounts and frequency, e.g., dosages. The communication means is useful for instructing on the benefits of using the present invention and communicating the approved methods for administering the invention to an animal.

In another aspect, the present invention provides packages comprising one or more LCPUFAs or a material suitable for containing one or more LCPUFAs and a label affixed to the package containing a word or words, picture, design, acronym, slogan, phrase, or other device, or combination thereof, that indicates that the contents of the package contains compounds that are useful for increasing endogenous plasmalogen levels, preventing or treating diseases or conditions in an animal caused or affected by decreased plasmalogen levels, extending the healthy prime years of an animal's life, or promoting the health or wellness of an animal. Typically, such device comprises the words “increases endogenous plasmalogen levels” or “useful for preventing or treating diseases or conditions in an animal caused or affected by decreased plasmalogen levels” or an equivalent expression printed on the package. Any package or packaging material suitable for containing dog food is useful in the invention, e.g., a bag, box, bottle, can, pouch, and the like manufactured from paper, plastic, foil, metal, and the like. In one embodiment, the package further comprises one or more plasmalogen agents. In a preferred embodiment, the package contains a food composition comprising LCPUFAs and a plasmalogen agent, preferably for administration to a companion animal.

In another aspect, the invention provides compositions useful for increasing endogenous plasmalogen levels in an animal comprising one or more LCPUFAs in amounts sufficient for increasing endogenous plasmalogen levels in an animal. In various embodiments, the composition comprises LCPUFAs in amounts sufficient to administer LCPUFAs to the animal in amounts of from about 1 to about 1000 mg/kg/day, preferably from about 5 to about 500 mg/kg/day, most preferably from about 10 to about 300 mg/kg/day.

In various embodiments, the animal is a human or companion animal. Preferably, the companion animal is a canine such as a dog or a feline such as a cat. In one embodiment, the animal is an aging animal, particularly an aging animal suffering from or susceptible to diseases or conditions that are characteristic of aging.

### EXAMPLES

The invention can be further illustrated by the following examples, although it will be understood that these examples are included merely for purposes of illustration and are not intended to limit the scope of the invention unless otherwise specifically indicated.

### Example 1

Terminology. DHA means docosahexaenoic acid; DHA-TG means docosahexaenoic containing triacylglycerols; DHA-PL means docosahexaenoic containing phospholipids; DMA means dimethylacetate; and PE means phosphatidylethanolamine.

Eighteen (18) Sprague Dawley rats were mated for a period of ten (10) days under controlled conditions for light (lights on, 7:00 AM-7:00 PM), temperature (22±1° C.) and hygrometry (55-60%) and fed one of three (3) diets as shown in Table 1. The control diet did not contain DHA or other LCPUFAs while the DHA-TG and DHA-PL groups contained the same level of DHA added in these diets as triacylglycerols (fish oil) or phospholipids (Krill Oil). After a weaning period (21 days after birth), all the neonates were fed a DHA free diet.

TABLE 1
Diet Compositions (Grams per Kilogram of Diet)

| | | Diets provided to | | | Diet provided to the |
|---|---|---|---|---|---|
| | | the females during the | | | young rats after the |
| | | gestation and the lactation periods | | | weaning period |
| | | Control | DHA-TG | DHA-PL | Growing Diet |
| Lipids | | 200 | 200 | 200 | 50 |
| Incl. | 18:2n-6 | 34.12 | 33.12 | 32.68 | 35 |
| | 18:3n-3 | 6.44 | 3.46 | 3.34 | 7 |
| | DHA | — | 1.2 | 1.2 | — |

| | | | | |
|---|---|---|---|---|
| Casein | 270 | 270 | 270 | 180 |
| Starch | 200 | 200 | 200 | 460 |
| Glucose | 207.65 | 207.65 | 207.65 | 230 |
| Non nutritive | 50 | 50 | 50 | 20 |
| fiber | | | | |
| Vitamins (mix) | 10 | 10 | 10 | 10 |
| Minerals (mix) | 50.85 | 50.85 | 50.85 | 50 |
| L-methionine | 2.5 | 2.5 | 2.5 | — |
| Choline | 2.75 | 2.75 | 2.75 | — |
| Inositol | 6.25 | 6.25 | 6.25 | — |

Six (6) young male newborns have been sacrificed after 14 days, 21 days, and 3 months of life after receiving the Growing Diet. For each group, brain glial cells phospholipids including PE were fractionated by high-performance liquid chromatography. The fractions were submitted to methanolysis under acidic condition to convert, at the same time, the alkenyl chains found in plasmenylethanoline into DMA derivatives and the fatty acyl chains into fatty acid methyl esters. The DMA and fatty acid methyl esters were analyzed by gas-liquid chromatography. Plasmenylethanolamine levels were determined as their DMA derivatives by gas-liquid chromatography. The results are shown in Table 2.

TABLE 2
Level of Dimethylacetate (DMA) Derived from Plasmenylethanolamine
in Brain Glial Cell Phosphatidylethanolamine (PE)

| | | | | | | | | | DHA-TG vs |
|---|---|---|---|---|---|---|---|---|---|
| | Control | | DHA-TG | | | DHA-PL | | | DHA-PL |
| | MV | SD | MV | SD | P value¹ | MV | SD | P value¹ | P value² |
| Day 14 | 8.24 | 0.78 | 9.76 | 0.48 | <0.0001 | 9.41 | 0.39 | 0.0003 | >0.05 |
| Day 21 | 10.14 | 0.42 | 11.29 | 0.73 | 0.0004 | 12.44 | 0.32 | <0.0001 | 0.0004 |
| Month 3³ | 10.05 | 0.5 | 11.71 | 0.29 | <0.0001 | 12.28 | 0.56 | 0.0001 | >0.05 |

¹P value related to the statistical comparison with the control group
²P value related to the statistical comparison between DHA-TG and DHA-PL groups
³Rats have been sacrificed 3 months after birth and received after 21 days of life a diet deprived of DHA

Referring to Table 2, the results show that the levels of DMA derived from the PE plasmalogen sn-1 vinyl ether residues were higher in rat neonates from the n-3 LCPUFA groups. Both DHA diets significantly increased DMA levels in PE purified from brain glial cells in male rats at day 14, day 21, and 3 months after birth. At day 21, the level of DMA in the DHA-PC group was higher than in the DHA-TG group (P=0.0004). This shows that DHA-PC can be preferably used as a n-3 LCPUFA supplement. Further, supplementation with n-3 LCPUFAs was performed through the maternal diet and was stopped at the end of weaning. Measurement of brain glial cell plasmenylethanolamine 3 months after birth shows that the effect of maternal diet supplementation lasts for long periods.

### Example 2

Eighteen (18) male Sprague Dawley rats were maintained for a period of ten (10) days under controlled conditions for light (lights on, 7:00 AM-7:00 PM), temperature (22±1° C.) and hygrometry (55-60%) and fed one of three (3) diets as shown in Table 3.

TABLE 3
Diet Compositions (Grams per Kilogram of Diet)

| | | Experimental Diets | | |
|---|---|---|---|---|
| | | Control | DHA-TG | DHA-PL |
| Lipids | | 200 | 200 | 200 |
| Incl. | 18:2n-6 | 34.12 | 33.12 | 32.68 |
| | 18:3n-3 | 6.44 | 3.46 | 3.34 |
| | DHA | — | 1.2 | 1.2 |

| | | | |
|---|---|---|---|
| Casein | 270 | 270 | 270 |
| Starch | 200 | 200 | 200 |
| Glucose | 207.65 | 207.65 | 207.65 |
| Non nutritive fiber | 50 | 50 | 50 |
| Vitamins (mix) | 10 | 10 | 10 |
| Minerals (mix) | 50.85 | 50.85 | 50.85 |
| L-methionine | 2.5 | 2.5 | 2.5 |
| Choline | 2.75 | 2.75 | 2.75 |
| Inositol | 6.25 | 6.25 | 6.25 |

After ten (10) days, the animals were euthanized and the composition of brain glial cell phosphatidylethanolamine was determined by gas-liquid chromatography. The results are show in Table 4.

TABLE 4
Level of Dimethylacetate (DMA) Derived from Plasmenylethanolamine
in Brain Glial Cell Phosphatidylethanolamine (PE) in Adult
Rats after 10 Days of Supplementation

| | | | | | | | | | DHA-TG vs |
|---|---|---|---|---|---|---|---|---|---|
| | Control | | DHA-TG | | | DHA-PL | | | DHA-PL |
| | MV | SD | MV | SD | P value¹ | MV | SD | P value¹ | P value² |
| Day 10 | 9.24 | 0.26 | 11.38 | 1.39 | <0.0001 | 12.04 | 0.14 | <0.0001 | >0.05 |

¹P value related to the statistical comparison with the control group
²P value related to the statistical comparison between DHA-TG and DHA-PL groups

Referring to Table 4, the results show that short term dietary supplementation with two different types of n-3 LCPUFAs supplements increases the level of plasmenylethanolamine, measured as DMA, in brain glial cell in adult animals.

### Example 3

Eighteen (18) female Sprague Dawley rats were fed for a period of forty-two (42) days under controlled conditions for light (lights on, 7:00 AM-7:00 PM), temperature (22±1° C.) and hygrometry (55-60%) and fed one of three (3) diets as shown in Table 5.

TABLE 5
Diet Compositions (Grams per Kilogram of Diet)

| | | Experimental Diets | | |
|---|---|---|---|---|
| | | Control | DHA-TG | DHA-PL |
| Lipids | | 200 | 200 | 200 |
| Incl. | 18:2n-6 | 34.12 | 33.12 | 32.68 |
| | 18:3n-3 | 6.44 | 3.46 | 3.34 |
| | DHA | — | 1.2 | 1.2 |

| | | | |
|---|---|---|---|
| Casein | 270 | 270 | 270 |
| Starch | 200 | 200 | 200 |
| Glucose | 207.65 | 207.65 | 207.65 |
| Non nutritive fiber | 50 | 50 | 50 |
| Vitamins (mix) | 10 | 10 | 10 |
| Minerals (mix) | 50.85 | 50.85 | 50.85 |
| L-methionine | 2.5 | 2.5 | 2.5 |
| Choline | 2.75 | 2.75 | 2.75 |
| Inositol | 6.25 | 6.25 | 6.25 |

After forty-two (42) days, the animals were euthanized and the composition of brain glial cell PE was determined by gas-liquid chromatography. The results are show in Table 6.

TABLE 6
Level of Dimethylacetate (DMA) Derived from Plasmenylethanolamine
in Brain Glial Cell Phosphatidylethanolamine (PE) in Adult
Rats after 42 Days of Supplementation

| | | | | | | | | | DHA-TG vs |
|---|---|---|---|---|---|---|---|---|---|
| | Control | | DHA-TG | | | DHA-PL | | | DHA-PL |
| | MV | SD | MV | SD | P value¹ | MV | SD | P value¹ | P value² |
| Day 42 | 17.37 | 0.12 | 19.05 | 0.13 | <0.0001 | 19.29 | 0.42 | <0.0001 | >0.05 |

¹P value related to the statistical comparison with the control group
²P value related to the statistical comparison between DHA-TG and DHA-PL groups

Referring to Table 6, the results show that long term dietary supplementation with two different types of n-3 LCPUFAs supplements increases the level of plasmenylethanolamine, measured as DMA, in brain glial cell in adult animals.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. A method for increasing endogenous plasmalogen levels in an animal comprising administering to the animal an endogenous plasmalogen level increasing amount of one or more long chain polyunsaturated fatty acids (LCPUFAs).

2. The method of claim 1 wherein the LCPUFAs are monocarboxylic acids having at least 18 carbon atoms and at least two double bonds.

3. The method of claim 1 wherein the LCPUFAs are omega-6 fatty acids and omega-3 fatty acids.

4. The method of claim 1 wherein the LCPUFAs are arachidonic acid, eicosapentaenoic acid, docosapentaenoic acid, and docosahexaenoic acid.

5. The method of claim 1 wherein the LCPUFAs are administered as LCPUFAs esters.

6. The method of claim 5 wherein the esters are acylglycerols or glycerophospholipids.

7. The method of claim 1 wherein the LCPUFAs are administered orally.

8. The method of claim 1 wherein the LCPUFAs are administered to the animal in amounts of from about 1 to about 1000 mg/kg/day.

9. (canceled)

10. (canceled)

11. The method of claim 1 wherein the LCPUFAs are administered to the animal in a diet in amounts of from about 1 to about 1000 g/kg/diet.

12. (canceled)

13. (canceled)

14. (canceled)

15. (canceled)

16. (canceled)

17. (canceled)

18. A method for preventing or treating a disease or condition in an animal caused or affected by decreased plasmalogen levels comprising administering an endogenous plasmalogen level increasing amount of one or more long chain polyunsaturated fatty acids (LCPUFAs) to an animal susceptible to or suffering from the disease or condition.

19. The method of claim 18 wherein the disease or condition is metabolic syndrome, neurodegenerative disease, dementia, Alzheimer's disease, cognitive impairment, colon cancer, prostate cancer, lung cancer, breast cancer, ovarian cancer, and kidney cancer.

20. The method of claim 18 wherein the LCPUFAs are monocarboxylic acids having at least 18 carbon atoms and at least two double bonds.

21. The method of claim 18 wherein the LCPUFAs are omega-6 acids and omega-3 fatty acids.

22. The method of claim 18 wherein the LCPUFAs are arachidonic acid, eicosapentaenoic acid, docosapentaenoic acid, and docosahexaenoic acid.

23. The method of claim 18 wherein the LCPUFAs are administered as LCPUFAs esters.

24. The method of claim 23 wherein the esters are acylglycerols or glycerophospholipids.

25. The method of claim 18 wherein the LCPUFAs are administered orally.

26. The method of claim 18 wherein the LCPUFAs are administered to the animal in amounts of from about 1 to about 1000 mg/kg/day.

27. (canceled)

28. (canceled)

29. The method of claim 18 wherein the LCPUFAs are administered to the animal in a diet in amounts of from about 1 to about 1000 g/kg/diet.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A method for promoting the health or wellness of an animal comprising administering to the animal an endogenous plasmalogen level increasing amount of one or more long chain polyunsaturated fatty acids (LCPUFAs).

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)
